(19) United States
(12) Patent Application Publication
(10) Pub. No.: US 2011/0015374 A1
(43) Pub. Date: Jan. 20, 2011

(54) Method for Commercial Isolation of Egg Yolk IgY, Its Product, and Uses of the Product

(75) Inventors: David John MASON, North Vancouver (CA); John Hugh M. MASON, West Vancouver (CA); Ian COOKE, Chilliwack (CA); James CAMPBELL, Abbotsford (CA)

Correspondence Address: STEPHEN R. BURRI, MILLER THOMSON LLP, 1000 - 840 HOWE STREET, VANCOUVER, BC, V6Z 2M1, CA

(73) Assignee: IRI SEPARATION TECHNOLOGIES INC., Vancouver, BC (CA)

(21) Appl. No.: 12/504,248

(22) Filed: Jul. 16, 2009

(51) Int. Cl. C07K 16/00; C07K 1/18

(52) U.S. Cl. 530/387.1; 530/416

(57) ABSTRACT

A method for extraction and purification of IgY from immune avian egg yolk comprising diluting a mixture of liquid egg yolk and liquid egg white in treated water; stirring the diluted mixture to obtain an egg mixture; separating the egg mixture into a first water soluble fraction, an unseparated mixture fraction, and a first separated lipophilic fraction;

separating the first water soluble fraction; the unseparated mixture fraction, and the first separated lipophilic fraction; centrifuging the unseparated mixture fraction until it separates into a second water soluble fraction and a second separated lipophilic fraction; adding the second water soluble fraction to the first water soluble fraction to form a combined water soluble fraction; passing the combined water soluble fraction through an ion exchange adsorption column containing IgY-adsorbent beads to adsorb IgY; eluting the adsorbed IgY of from the ion exchange adsorption column with an eluting solution to obtain an eluant; filtering the eluant of through a first membrane filtration system to obtain a 4% to 25% IgY concentrate; and drying the IgY concentrate to obtain a 40% to 98% IgY isolate, as well as a food-safe IgY isolate obtained and use of a food-safe IgY isolate in an edible product.

### FIELD OF THE INVENTION

The present invention relates to a method for isolation of egg yolk immunoglobulins. In particular, the present invention relates to a method for isolation of highly purified immunoglobulins from avian egg yolk on a commercial scale.

### BACKGROUND OF THE INVENTION

Egg yolk separated from eggs obtained from poultry is an excellent source of polyclonal antibodies. For example, immunoglobulin Y (‘IgY’) may be isolated from chicken egg yolk.

There are several advantages to obtaining immunoglobulins from chicken egg yolks. Use of egg yolk does not require blood extraction, as is the case with mammalian antibody sources, so it may be easier to comply with animal protection regulations. Chicken eggs also provide a continual supply of source material. The extent of non-specific binding by IgY is reduced, relative to immunoglobulin sourced from mammals, as chicken IgY is not cross-reactive with mammalian IgG. Avian-produced immunoglobulins are potentially more useful in mammalian species than are mammal-produced immunoglobulins, due to the phylogenetic distance between birds and mammals.

IgY has been orally administered successfully for treatment of several gastrointestinal infections, including human rotaviruses, enterotoxigenic Escherichia coli, and other infections caused by Salmonella, Staphyloccocus and Pseudomonas species. It also may be used to convey passive immunity to humans and other animals.

Many prior art methods of immunoglobulin isolation involve precipitation and the use of precipitating agents, such as ethanol, polyethylene glycol, lyotropic salts such as copper sulphate, ammonium sulphate and ammonium phosphate, and caprylic acid. The prior art does not teach a commercial scale method of immunoglobulin isolation from egg yolks which does not require such chemical agents, nor an IgY product which is free of the residues of such chemical agents.

While it is known in the art to provide a process for isolation of highly pure IgY from chicken egg yolk in a laboratory setting, the potential uses for IgY require a process for such isolation which is simple, cost-effective and may be used on a commercial scale.

### SUMMARY OF THE INVENTION

It is an object of the present invention to provide a method for commercial scale isolation of highly purified immunoglobulin Y from large volumes of chicken egg yolk. Such a method ideally should only incorporate chemicals permitted for use as a food or other edible product. It is a further object to provide a highly purified IgY isolate obtained by such a process. Other objects include providing a highly purified IgY isolate for use in edible products, including food, animal feed and dietary supplements.

According to one embodiment of a method of the invention, there is provided a method for extraction and purification of IgY from immune avian egg yolk comprising the following steps: separating the egg yolk from the albumen in a manner which provides a source of egg containing high solids egg yolk with a solids content of between 23% and 50%; diluting the high solids egg yolk obtained from 3-fold to 15-fold in treated water having a dilution temperature between 0.5 and 8° C. to obtain diluted egg yolk; stirring the diluted egg yolk obtained at a stirring temperature of between 0.5 and 8° C. until it is mixed to obtain an egg yolk mixture; allowing the egg yolk mixture obtained to settle in a settling container for a settling time of between 16 and 96 hours at a settling temperature of between 0.5 and 8° C., until the mixture separates into a water soluble fraction and a precipitated lipophilic fraction; separating the water soluble fraction and the precipitated lipophilic fraction; concentrating the water soluble fraction through a first membrane filtration system to obtain a filtered material; passing the filtered material through an ion exchange adsorption column containing IgY-adsorbent beads to adsorb IgY; eluting the adsorbed IgY from the ion exchange adsorption column with an eluting solution to obtain an eluant; filtering the eluant through a second membrane filtration system to obtain a 4% to 25% IgY concentrate; and drying the IgY concentrate of step (i) to obtain a 40% to 98% IgY isolate.

The solids content of the egg yolk may be at least 44%, preferably at least 46.5% solids. Each of the dilution, stirring and settling temperatures may be between 3 and 5° C., optimally 4° C. The settling time may be 36 hours. The eluting solution may be a sodium ion containing solution.

According to another embodiment of a method of the invention, there is provided a method for extraction and purification of IgY from immune avian egg yolk comprising the following steps: diluting a mixture of liquid egg yolk and liquid egg white from 3-fold to 15-fold in treated water having a temperature between 0.5 and 8° C.; stirring the diluted mixture obtained at a stirring temperature of between 0.5 and 8° C. until it may be mixed to obtain an egg mixture; separating the egg mixture into a first water soluble fraction, an unseparated mixture fraction, and a first separated lipophilic fraction; separating the first water soluble fraction; the unseparated mixture fraction, and the first separated lipophilic fraction; centrifuging the unseparated mixture fraction at a centrifugation force of between 1500 and 10,000 g for a centrifugation time of between 1 and 120 minutes until it separates into a second water soluble fraction and a second separated lipophilic fraction; adding the second water soluble fraction to the first water soluble fraction to form a combined water soluble fraction; passing the combined water soluble fraction through an ion exchange adsorption column containing IgY-adsorbent beads to adsorb IgY; eluting the adsorbed IgY of from the ion exchange adsorption column with an eluting solution to obtain an eluant; filtering the eluant of through a first membrane filtration system to obtain a 4% to 25% IgY concentrate; and drying the IgY concentrate to obtain a 40% to 98% IgY isolate.

Each of the dilution, stirring and settling temperatures may be between 3 and 5° C., optimally 4° C. The settling time may be 36 hours. The eluting solution may be a sodium ion containing solution. The centrifugation time may be between 1 and 15 minutes. The centrifugation force may be 3000 g.

The method may include a step after the ion exchange adsorption step of concentrating the water soluble fraction using a second membrane filtration system, and it may include a step after the elution step of concentrating the water soluble fraction using a third membrane filtration system.

The first separated lipophilic fraction may be combined with the second precipitate lipophilic fraction and the resultant combined precipitate lipophilic fraction may be further processed to isolate commercially useful components thereof.

According to a further embodiment of the present invention, there is provided a method for extraction and purification of IgY from immune avian egg yolk comprising the following steps: diluting a mixture of liquid egg yolk and liquid egg white from 3-fold to 15-fold in treated water having a dilution temperature between 0.5 and 8° C.; centrifuging the mixture at a centrifugation force of between 1500 and 10,000 g for a centrifugation time of between 1 and 120 minutes until it separates into a water soluble fraction and a separated lipophilic fraction; separating the water soluble fraction and the precipitated lipophilic fraction; passing the water soluble fraction through an ion exchange adsorption column containing IgY-adsorbent beads to adsorb IgY; eluting the adsorbed IgY from the ion exchange adsorption column with an eluting solution; filtering the eluant through a first membrane filtration system to obtain a 4% to 25% IgY concentrate; and drying the IgY concentrate to obtain a 40% to 98% IgY isolate.

Each of the dilution, stirring and settling temperatures may be between 3 and 5° C., optimally 4° C. The settling time may be 36 hours. The eluting solution may be a sodium ion containing solution. The centrifugation time may be between 1 and 15 minutes. The centrifugation force may be 3000 g.

The method may include a step after the ion exchange adsorption step of concentrating the water soluble fraction using a second membrane filtration system, and it may include a step after the elution step of concentrating the water soluble fraction using a third membrane filtration system.

According to another embodiment of the present invention, there is provided an IgY isolate obtained by the methods described herein. The IgY isolate may be a food-safe IgY isolate.

According to another aspect of the present invention, there is provided the use of a food-safe IgY isolate in an edible product.

In another aspect, the invention provides an IgY-rich isolate obtained by the method described herein. In a further aspect, the invention provides a use of the IgY-rich isolate obtained by the method described herein in an edible food product. In still a further aspect, the invention provides a method of treatment of acne comprising administration to a patient having acne a therapeutic amount of IgY-rich product obtained by the process described herein. In another of its aspects, the invention provides a method of passive immunization against pylori bacteria comprising administering to a patient an amount of IgY-rich product obtained by the process described herein sufficient to passively immunize the patient.

The IgY-rich isolate obtained by the method of the present invention does not require extraction additives such as fatty acids or detergents. It is safe for human or animal consumption, that is, it is edible. The IgY-rich isolate is free of any chemical residues.

### BRIEF DESCRIPTION OF THE DRAWING

The invention will be better understood when consideration is given to the following detailed description thereof. Such description makes reference to the annexed drawings wherein:

FIG. 1 is a schematic showing one embodiment of the process of the present invention;

FIG. 2 is a schematic showing an alternate embodiment of the process of the present invention; and

FIG. 3 is a schematic showing another embodiment of the process of the present invention.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE PREFERRED EMBODIMENT

In this specification, the following terms have the meanings set out below:

‘avian’ means poultry and fowl, including chickens, turkeys, geese, and ducks;

‘edible’ means not harmful for humans or animals when consumed, and includes food, animal feed, and dietary supplements;

‘edible product’ includes a pill, capsule, tablet, lozenge or other composition for nutritional use, illness prevention or therapeutic use;

‘food-safe IgY isolate’ means an IgY isolate having no residues of synthetic solvents, synthetic salts or other synthetic chemical substances added during the process of isolation of the IgY isolate;

‘high solids egg yolk’ means egg yolk having 44% to 50% solids;

‘IgY’ includes all immunoglobulins normally found in avian egg yolk;

‘expanded bed adsorption chromatography’ means the method of fractionation of a protein-containing solution described in patent applications WO 98/08603, WO 02/096215 and WO 04/082397, and is a method of ion exchange adsorption chromatography;

‘membrane filtration’ includes microfiltration, ultrafiltration, nanofiltration, reverse osmosis or similar filtration systems;

‘n-fold dilution’ of substance x means dilution of 1 part substance x with n parts treated water; and

‘synthetic solvents, synthetic salts or other synthetic chemical substances’ includes ammonium sulphate, ammonium phosphate, copper sulphate, caprylic acid and polyethylene, and excludes citric acid, hydrogen chloride, sodium hydroxide, sodium citrate, other sodium ion containing solutions, and resins used in ion exchange adsorption chromatography.

The present invention provides a process for extracting immunoglobulins from avian eggs. In particular, the process of the invention describes a method of isolation of immunoglobulin Y from egg yolk from immunized or non-immunized chickens. Vaccination and handling of immunized or non-immunized chickens are well known in the art.

In order to optimize the purity of IgY obtained, it is desirable to obtain an egg yolk source material which is high in solids. Pure chicken egg yolk typically contains more than 50% solids. Egg yolks separated individually in a lab may be blotted with an absorbing towel, increasing the solids to as much as 50%. As egg yolk separation from albumen on a commercial scale results in contamination of the egg yolk by the albumen, which has only about 12% solids composition, commercial egg yolk is only required to contain at least 43% solids. Commercial egg yolk may contain as much as 20% albumen.

Albumen contains overtransferrin, which is indistinguishable from IgY by the isolation process of the present invention. Albumen may also affect yields through adsorption of albumin protein to column beads in the ion exchange adsorption process step described below. To reduce overtransferrin contamination and albumin adsorption, and thus achieve more highly purified IgY, it is important to use a starting material which is as low in albumen as possible, at least 23% to 50% solids, preferably between 44% and 50% solids, and most preferably at least 46.5% solids and containing significantly less than 20% albumen. High solids egg yolk may be best obtained from fresh eggs using an in-line breaking process.

The egg yolk source material may be heat-treated. Such heat-treatment may comprise pasteurization.

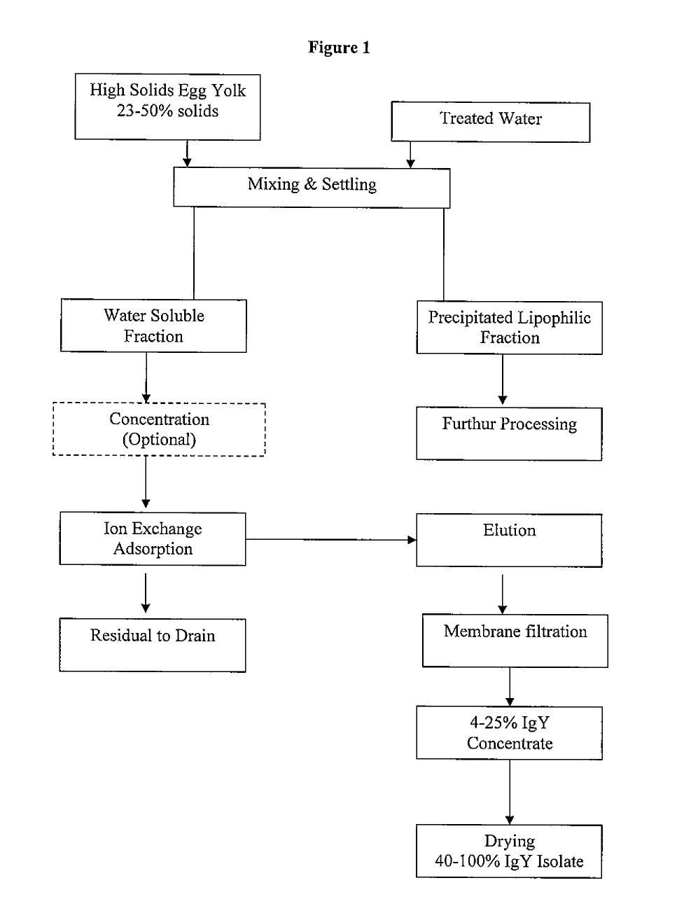

As indicated schematically in FIG. 1, according to one embodiment of a settling process of the present invention, one part high solids egg yolk may be mixed with nine parts treated water at a temperature of 0.5 to 8.0° C., preferably 3.0 to 5.0° C., optimally 4.0° C. Any dilution from 3-fold to 15-fold may be used, but a 9-fold dilution is optimal. The pH of the mixture may be adjusted if desired. The water may be purified by a reverse osmosis process to deionize and demineralized the water. Other water treatment procedures may instead be used.

The mixture may be stirred to permit mixing and to encourage precipitation of egg solids. The mixture may be permitted to settle at a temperature of 0.5 to 8.0° C., preferably 3.0 to 5.0° C., optimally 4.0° C., for at least 16 hours and for as long as 96 hours, optimally 36 hours, during which a phase separation into a water soluble fraction (“WSF”) rich in IgY and a precipitated lipophilic fraction (“PLF”) occurs.

The WSF and the PLF may be separated by pouring off, siphoning or similar method. The WSF may be concentrated through the use of membrane filtration according to a process well known in the art, to obtain a 5% to 7% solids fraction.

This 5% to 7% solids fraction may be run through an ion exchange adsorption column, which may be an expanded bed adsorption column, to isolate and purify the IgY. Ion exchange adsorption technology is known to persons skilled in the art, and is taught, for example in WO 92/00799, WO 92/18237, WO 97/17132, WO 98/33572, WO 00/57982, WO 01/58924. One method of ion exchange adsorption chromatography of the present invention is described in WO 98/08603, WO 02/096215 and WO 04/082397.

The IgY may be eluted from the adsorbent using a sodium ion containing solution or other eluting solution. The resulting eluant is concentrated and purified through a process of membrane filtration to obtain a 4% to 25% IgY concentrate. Finally, the purified and concentrated IgY concentrate is dried to obtain a 40% to 98% pure IgY isolate.

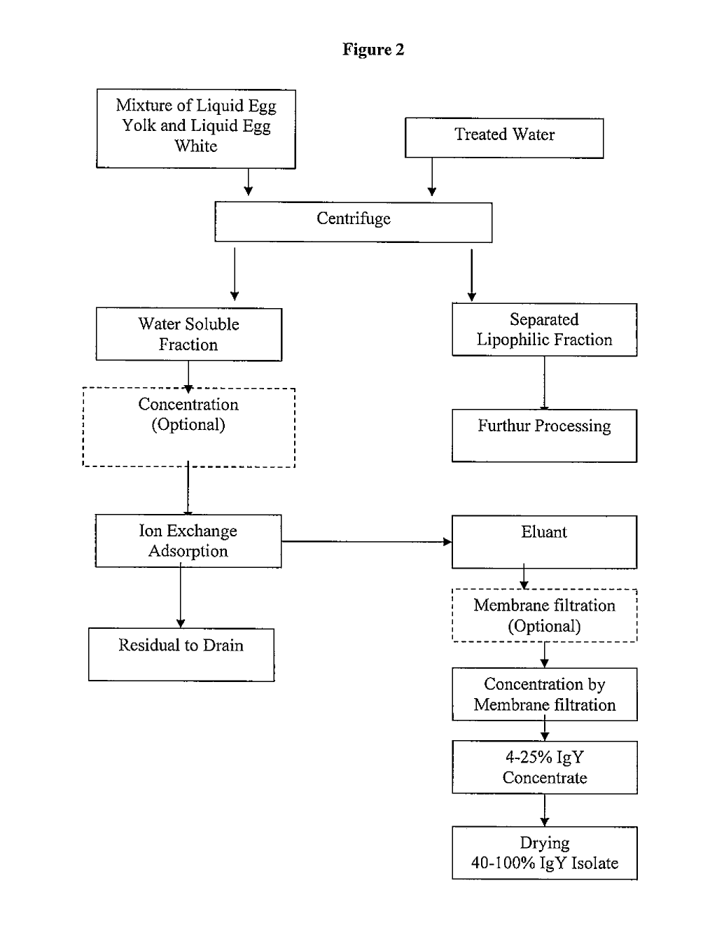

As depicted schematically in FIG. 2, according to an embodiment of a centrifugation process of the present invention, a mixture of liquid egg yolk and liquid egg white may be combined with treated water. Any dilution from 3-fold to 15-fold may be used, but a 9-fold dilution is optimal. Optionally, the pH of the mixture may be adjusted using sodium citrate, hydrogen chloride, citric acid or sodium hydroxide.

The mixture is centrifuged, preferably at a temperature of 0.5 to 8.0° C., more preferably 3.0 to 5.0° C., optimally 4.0° C., during which a phase separation into a WSF rich in IgY and a separated lipophilic fraction (“SLF”) occurs.

The WSF and the SLF may be separated by pouring off, siphoning or similar method. Optionally, the WSF may be concentrated by membrane according to a process well known in the art, to obtain a solids fraction.

This solids fraction, may be run through an ion exchange adsorption column, which may be an expanded bed adsorption column, to isolate and purify the IgY. The IgY may be eluted from the adsorbent using a sodium ion containing solution or equivalent. The resulting eluant optionally may be membrane filtered. The eluant may then be concentrated and purified through a process of membrane filtration to obtain a 4% to 25% IgY concentrate. The purified and concentrated IgY concentrate may be dried to obtain a 40% to 98% pure IgY isolate.

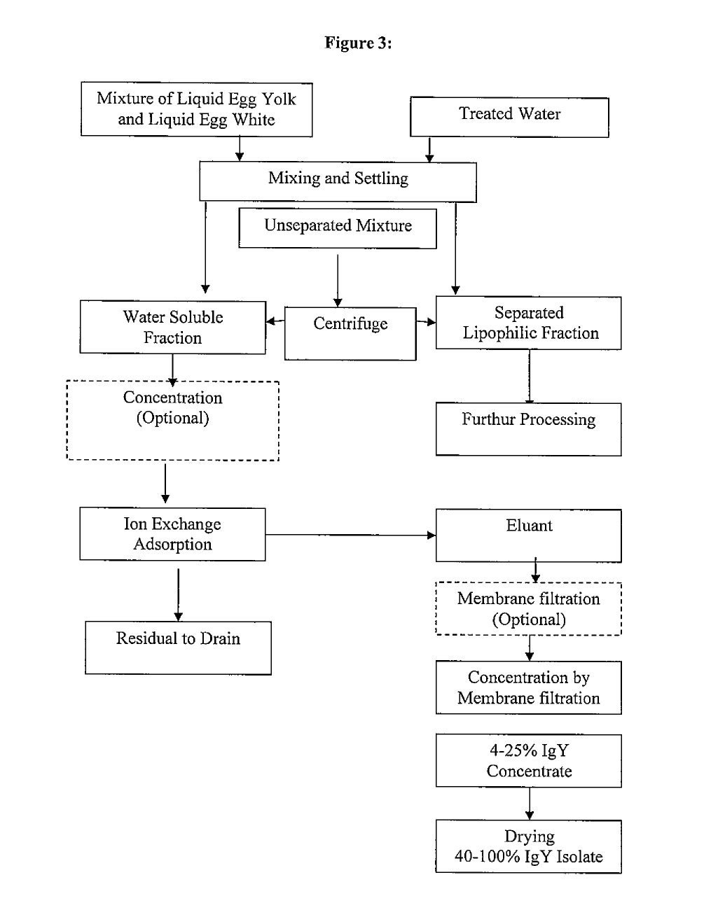

As depicted schematically in FIG. 3, according to another embodiment of a centrifugation process of the present invention, a mixture of liquid egg yolk and liquid egg white may be combined with treated water. Preferably the mixture may be maintained at a temperature of 0.5 to 8.0° C., more preferably 3.0 to 5.0° C., optimally 4.0° C. Any dilution from 3-fold to 15-fold may be used, but a 9-fold dilution is optimal. The pH of the mixture may be adjusted if desired.

The mixture may be stirred to permit mixing and to encourage precipitation of egg solids. The mixture may be permitted to settle at a temperature of 0.5 to 8.0° C., preferably 3.0 to 5.0° C., optimally 4.0° C., for at least 16 hours and for as long as 96 hours, optimally 36 hours, during which a phase separation into a first WSF rich in IgY, an unseparated mixture fraction (“UMF”), and a first SLF occurs.

The first WSF, the UMF and the first SLF may be separated by pouring off, siphoning or similar method. The UMF may be centrifuged at an RCF of 1500 to 10,000 g, optimally 3000 g, to effect a second phase separation into a second WSF rich in IgY and a second SLF. The second WSF may be removed by pouring off, siphoning or similar method and combined with the first WSF to form a combined WSF. Optionally, the combined WSF may be concentrated by membrane filtration to obtain a 5% to 7% solids fraction. The second SLF may be combined with the first SLF to form a combined SLF. The combined SLF may be further processed.

This 5% to 7% solids fraction may be run through an ion exchange adsorption column, which may be an expanded bed adsorption column, to isolate and purify the IgY. The IgY may be eluted from the adsorbent using a sodium ion containing solution or other eluting solution, with residual material in the adsorption bed directed to a drain. The resulting eluant optionally may be membrane filtered, and may then be concentrated and purified through a process of membrane filtration to obtain a 4% to 25% IgY concentrate. The purified and concentrated IgY concentrate may be dried to obtain a 40% to 98% pure IgY isolate.

The resulting IgY isolate comprises 40% to 98% avian immunoglobulin. The yield, concentration and purity of the IgY isolate are measurable by standard methods familiar to those skilled in the art. The resulting IgY isolate may be used in an edible product or as an edible product-additive.

It will be appreciated by those skilled in the art that other variations of the preferred embodiment may also be practiced without departing from the scope of the invention.

What is claimed is:

1. A method for extraction and purification of IgY from immune avian egg yolk comprising the following steps:
(a) separating the egg yolk from the albumen in a manner which provides a source of egg containing high solids egg yolk with a solids content of between 23% and 50%;
(b) diluting the high solids egg yolk obtained in step (a) from 3-fold to 15-fold in treated water having a dilution temperature between 0.5 and 8° C. to obtain diluted egg yolk;
(c) stirring the diluted egg yolk obtained from step (b) at a stirring temperature of between 0.5 and 8° C. until it is mixed to obtain an egg yolk mixture;
(d) allowing the egg yolk mixture obtained from step (c) to settle in a settling container for a settling time of between 16 and 96 hours at a settling temperature of between 0.5 and 8° C., until the mixture separates into a water soluble fraction and a precipitated lipophilic fraction;
(e) separating the water soluble fraction and the precipitated lipophilic fraction from step (d);
(f) concentrating the water soluble fraction of step (e) through a first membrane filtration system to obtain a filtered material;
(g) passing the filtered material of step (f) through an ion exchange adsorption column containing IgY-adsorbent beads to adsorb IgY;
(h) eluting the adsorbed IgY of step (g) from the ion exchange adsorption column with an eluting solution to obtain an eluant;
(i) filtering the eluant of step (h) through a second membrane filtration system to obtain a 4% to 25% IgY concentrate; and
(j) drying the IgY concentrate of step (i) to obtain a 40% to 98% IgY isolate.

2. The method of claim 1 wherein the solids content of the egg yolk is at least 44%.

3. The method of claim 2 wherein the solids content of the egg yolk is at least 46.5% solids.

4. The method of claim 1, wherein each of the dilution, stirring and settling temperatures is between 3 and 5° C.

5. The method of claim 3, wherein each of the dilution, stirring and settling temperatures is 4° C.

6. The method of claim 1, wherein the settling time is 36 hours.

7. The method of claim 1, wherein the eluting solution is a sodium ion containing solution.

8. A method for extraction and purification of IgY from immune avian egg yolk comprising the following steps:
(a) diluting a mixture of liquid egg yolk and liquid egg white from 3-fold to 15-fold in treated water having a temperature between 0.5 and 8° C.;
(b) stirring the diluted mixture obtained from step (a) at a stirring temperature of between 0.5 and 8° C. until it is mixed to obtain an egg mixture;
(c) separating the egg mixture of step (b) into a first water soluble fraction, an unseparated mixture fraction, and a first separated lipophilic fraction;
(d) separating the first water soluble fraction; the unseparated mixture fraction, and the first separated lipophilic fraction;
(e) centrifuging the unseparated mixture fraction at a centrifugation force of between 1500 and 10,000 g for a centrifugation time of between 1 and 120 minutes until it separates into a second water soluble fraction and a second separated lipophilic fraction;
(f) adding the second water soluble fraction to the first water soluble fraction to form a combined water soluble fraction;
(g) passing the combined water soluble fraction of step (f) through an ion exchange adsorption column containing IgY-adsorbent beads to adsorb IgY;
(h) eluting the adsorbed IgY of step (g) from the ion exchange adsorption column with an eluting solution to obtain an eluant;
(i) filtering the eluant of step (h) through a first membrane filtration system to obtain a 4% to 25% IgY concentrate;
(j) drying the IgY concentrate of step (i) to obtain a 40% to 98% IgY isolate.

9. The method of claim 8, wherein each of the dilution, stirring and settling temperatures is between 3 and 5° C.

10. The method of claim 9, wherein each of the dilution, stirring and settling temperatures is 4° C.

11. The method of claim 8, wherein the centrifugation time is between 1 and 15 minutes.

12. The method of claim 8, wherein the centrifugation force is 3000 g.

13. The method of claim 8 further comprising a step after the ion exchange adsorption step of concentrating the water soluble fraction using a second membrane filtration system.

14. The method of claim 8, wherein the eluting solution is a sodium ion containing solution.

15. The method of claim 8 further comprising a step after the elution step of concentrating the water soluble fraction using a third membrane filtration system.

16. The method of claim 8, wherein the first separated lipophilic fraction of step (c) is combined with the second precipitate lipophilic fraction of step (e) and the resultant combined precipitate lipophilic fraction is further processed to isolate commercially useful components thereof.

17. A method for extraction and purification of IgY from immune avian egg yolk comprising the following steps:
(a) diluting a mixture of liquid egg yolk and liquid egg white from 3-fold to 15-fold in treated water having a dilution temperature between 0.5 and 8° C.;
(b) centrifuging the mixture of step (a) at a centrifugation force of between 1500 and 10,000 g for a centrifugation time of between 1 and 120 minutes until it separates into a water soluble fraction and a separated lipophilic fraction;
(c) separating the water soluble fraction and the precipitated lipophilic fraction from step (b);
(d) passing the water soluble fraction of step (c) through an ion exchange adsorption column containing IgY-adsorbent beads to adsorb IgY;
(e) eluting the adsorbed IgY of step (d) from the ion exchange adsorption column with an eluting solution;
(f) filtering the eluant of step (e) through a first membrane filtration system to obtain a 4% to 25% IgY concentrate;
(g) drying the IgY concentrate of step (f) to obtain a 40% to 98% IgY isolate.

18. The method of claim 17, wherein the dilution temperature is between 3 and 5° C.

19. The method of claim 18, wherein the dilution temperature is 4° C.

20. The method of claim 17, wherein the centrifugation time is between 1 and 15 minutes.

21. The method of claim 17, wherein the centrifugation force is 3000 g.

22. The method of claim 17 further comprising a step (c1) after step (c) of concentrating the water soluble fraction using a second membrane filtration system.

23. The method of claim 17, wherein the eluting solution is a sodium ion containing solution.

24. The method of claim 17 further comprising a step (e1) after step (e) of concentrating the water soluble fraction using a third membrane filtration system.

25. An IgY isolate obtained by the method of claim 1.

26. The IgY isolate of claim 25, wherein the IgY isolate is a food-safe IgY isolate.

27. The use of the food-safe IgY isolate of claim 26 in an edible product.

28. An IgY isolate obtained by the method of claim 8.

29. The IgY isolate of claim 28, wherein the IgY isolate is a food-safe IgY isolate.

30. The use of the food-safe IgY isolate of claim 29 in an edible product.

31. An IgY isolate obtained by the method of claim 17.

32. The IgY isolate of claim 31, wherein the IgY isolate is a food-safe IgY isolate.

33. The use of the food-safe IgY isolate of claim 32 in an edible product.
